(19) United States
(12) Patent Application Publication
(10) Pub. No.: US 2020/0131901 A1
(43) Pub. Date: Apr. 30, 2020

(54) SHALE GAS ENRICHMENT AND ACCUMULATION CLASSIFICATION METHOD

(71) Applicant: SOUTHWEST PETROLEUM UNIVERSITY, Chengdu (CN)

(72) Inventors: Chenghua OU, Chengdu (CN); Chaochun LI, Chengdu (CN)

(73) Assignee: SOUTHWEST PETROLEUM UNIVERSITY, Chengdu (CN)

(21) Appl. No.: 16/315,151

(22) PCT Filed: Sep. 15, 2017
(86) PCT No.: PCT/CN2017/101834

§ 371 Date: Jan. 3, 2019

(30) Foreign Application Priority Data
Sep. 11, 2017 (CN) 201710811197.0

(51) Int. Cl. E21B 49/00; G01N 33/24

(52) U.S. Cl. CPC E21B 49/00; G01N 33/241

(57) ABSTRACT

A shale gas enrichment and accumulation classification method includes the following steps: S1, analyzing a shale gas enrichment and accumulation system to form shale gas enrichment and accumulation modes of different degrees; S2, dividing the shale gas enrichment and accumulation modes into a tectonic main control hydrocarbon generation source rock type shale gas enrichment and accumulation mode, a tectonic main control gas accumulation reservoir-type shale gas enrichment and accumulation mode and a tectonic main control protective stratum type enrichment and accumulation mode according to the differences of the degrees of structure evolution over shale gas hydrocarbon generation source rock, a gas accumulation reservoir and a protective stratum; and S3, dividing the tectonic main control hydrocarbon generation source rock type shale gas enrichment and accumulation mode into an autochthonous continuous biogenic shale gas enrichment and accumulation mode and an autochthonous thermogenic shale gas enrichment and accumulation mode.

### CROSS REFERENCE TO THE RELATED APPLICATIONS

This application is the national phase entry of International Application No. PCT/CN2017/101834, filed on Sep. 15, 2017, which is based upon and claims priority to Chinese Patent Application No. 201710811197.0 filed on Sep. 11, 2017, the entire contents of which are incorporated herein by reference.

### TECHNICAL FIELD

The present invention relates to the technical field of shale gas enrichment and accumulation classification, in particular to a shale gas enrichment and accumulation classification method.

### BACKGROUND

Because the shale matrix is dominated by nanopores, it is much denser than other fluid reservoirs, resulting in an enrichment and accumulation mode of shale gas that is significantly different from other fluid deposits. As early as 1995, the US Geological Survey introduced the concept of “continuous” oil and gas reservoirs in the US shale gas evaluation. In 2002, Curtis defined shale gas as a continuous gas reservoir. In 2005, the US Geological Survey clearly stated that shale gas belongs to a continuous enrichment and accumulation type. Then, the shale continuous accumulation theory has been introduced into China and has been promoted and applied in China's shale gas exploration practice.

The shale gas continuous enrichment and accumulation mode indicates that the shale generates biochemigenic gas, thermogenic gas, or a mixture gas thereof, characterized by hidden accumulation mechanism, short migration distance and various lithologic closures. The enrichment area of the shale gas is continuously distributed over a large area, and the gas reservoir boundary is limited only by the distribution of shale layers. The shale gas enrichment area is continuously distributed over a large area, and the gas reservoir boundary is limited only by the distribution of shale formation. It is considered in the shale gas continuous enrichment and accumulation mode that the shale is of both hydrocarbon source rock and accumulation rock, has relatively strong closure performance per se and belongs to a typical source-storage-cap integrated enrichment mode. This mode emphasizes the integration of the hydrocarbon generation source rock and the gas accumulation reservoir, places the role of reservoir protection at a secondary location, and ignores the separation of the hydrocarbon generation source rock and the gas accumulation reservoir caused by tectonic evolution, as well as the transformation and destruction to the gas accumulation reservoir and the protective stratum, while not emphasizing the matching degree of the temporal and spatial relationships of accumulation elements.

Although the result of Barnett shale gas enrichment is large-scale continuous distribution in pieces, in the enrichment and accumulation process, the tectonic evolution substantially forms a burial depth required for generation of a large amount of hydrocarbons, and a hydrocarbon drainage depth required for enrichment and accumulation. That is, the role and influence of tectonic evolution on its hydrocarbon generation and hydrocarbon drainage systems cannot be ignored. The spatial form of the gas accumulation reservoir of the shale gas reservoir in Jiaoshiba has been greatly modified by the tectonic evolution, resulting in the separation of the hydrocarbon generation source rock and the gas accumulation reservoir, such that the shale gas is not concentrated autochthonously, but is enriched and accumulated again at different locations on the same shale layer after a certain distance of migration. It can thus be seen that the existing shale gas continuous enrichment and accumulation theory cannot explain the discovery of more and more shale gas reservoirs with different enrichment characteristics, which is difficult to adapt to the needs of current further shale gas exploration and development. Therefore, there is an urgent need to establish a more elaborate shale gas enrichment and accumulation mode with various shale gas enrichment characteristics to guide the practical needs of exploration and development of various shale gases with different enrichment and accumulation characteristics.

### SUMMARY

An objective of the present invention is to overcome the defects of the prior art, and provide a shale gas enrichment and accumulation classification method which breaks through the original shale continuous accumulation theory and technique system, makes the classification more elaborate and provides a favorable support for further exploration and development of shale gas.

The objective of the present invention is implemented by the following steps: a shale gas enrichment and accumulation classification method comprises the following steps:

S1. analyzing a shale gas enrichment and accumulation system and dividing the same into three static subsystems of hydrocarbon generation source rock, a gas accumulation reservoir and a protective stratum, as well as four dynamic subsystems of tectonic evolution, sedimentary sequence, diagenetic evolution and hydrocarbon generation history, and establishing three major types, six sub-categories of shale gas enrichment and accumulation modes based on the analysis of the interaction relationship between the dynamic subsystems and the static subsystems and the extraction of principal factors;

S2, dividing the shale gas enrichment mode in the step S1 into a tectonic main control hydrocarbon generation source rock type shale gas enrichment mode, a tectonic main control gas accumulation reservoir-type shale gas enrichment mode and a tectonic main control protective stratum type shale gas enrichment mode according to the differences of the degrees of tectonic evolution over shale gas hydrocarbon generation source rock, a gas accumulation reservoir and a protective stratum;

S3, dividing the tectonic main control hydrocarbon generation source rock type shale gas enrichment mode into two sub-categories:

S3(I), an autochthonous continuous biogenic shale gas enrichment mode: a basin in which the tectonic subsidence extent is not large, the buried depth is low, pore water in mud shale is not completely discharged, and meanwhile organic matter-rich bud shale begins to generate biogenetic shale gas in anoxic, low-temperature and watery environments, and is accumulated autochthonously by means of continuous filling of atmospheric fresh water at the edge of the basin, is classified as the autochthonous continuous biogenic shale gas enrichment mode;

S3(II), an autochthonous thermogenic shale gas enrichment mode: a basin in which the tectonic subsidence extent increases, the buried depth increases, the formation temperature and pressure gradually increase, primary water in pores is gradually discharged by a compaction effect, gradually evaporates under the influence of high temperature and high pressure environments, and is finally exhausted, and kerogen and asphalt organic matters in mud shale begin to generate a large amount of hydrocarbons via thermal degradation or thermal cracking, is classified as the autochthonous thermogenic shale gas enrichment mode; S4, dividing the tectonic main control gas accumulation reservoir-type shale gas enrichment mode into the following two sub-categories:

S4(I), a positive tectonic accumulation reservoir-type shale gas enrichment mode: a forelandbasin in which, during the formation process, or during the generation and drainage of a large amount of hydrocarbons of hydrocarbon source rock after formation, an area where the gas accumulation reservoir is located has undergone strong tectonic compression, resulting in large fold deformation of the gas accumulation reservoir that is originally located in a monoclinic structure of the basin slope or in a negative structure in the basin center, is classified as the positive tectonic accumulation reservoir-type shale gas enrichment mode;

S4(II) a fractural zone accumulation type shale gas enrichment mode: a forelandbasin in which, during the formation process, or during the generation and drainage of a large amount of hydrocarbons of hydrocarbon source rock after formation, or at the end of the hydrocarbon generation or drainage process, multiple stages of tectonic lifting movements occur in an area where the gas accumulation reservoir is located, the burial depth of the gas accumulation reservoir results in a sharp fluctuation of the formation temperature and pressure caused by turbulent changes, gas adsorption and desorption processes and free gas shrinkage and expansion processes are repeated continuously to promote the activation of various diagenetic fractures in the gas accumulation reservoir, and the interactive changes of stress concentration formed by the tectonic lifting movements also induce more tectonic fractures, is classified as the fractural zone accumulation type shale gas enrichment mode;

S5, dividing a tectonic main control protective stratum type shale gas accumulation mode into the following two sub-categories:

S5(I), a fracture-damaged type shale gas accumulation mode: a shale gas reservoir in which, after undergoing multiple stages of tectonic compression or tensile actions, various types of compressed or tensile faults and induced fractures thereof begin to occur in the core area of the shale gas reservoir, the original gas accumulation reservoir and top and bottom protective strata thereof begin to be cut by many fault blocks, the shale gas enriched near the faults gradually dissipates to relief pressure along the faults and the induced fractures thereof, causing the cutting damage of the original shale gas reservoir, the shale itself is dense and has a certain covering capability, and in addition to the existence of the top and bottom protective layers, the locally accumulated shale gas remains in the fault blocks away from the faults and the induced fractures, is classified as the fracture-damaged type shale gas accumulation mode;

S(II), a denudation residual type shale gas accumulation mode: a shale gas reservoir in which, after undergoing multiple stages of tectonic uplifting movements, the core area of the shale gas reservoir will continue to rise, the dip angle of the formation becomes larger, causing the updraft ends of the gas accumulation reservoir and the top and bottom protective strata thereof to be exposed out of the earth surface and suffer from the leaching effect of surface atmospheric water, shale gas enriched therein is adsorbed to form a large amount of free gas because of depressurization and desorption, N2 and CO2 from air displaces a large amount of shale gas due to stronger adsorption after entering into the shale reservoir on the other hand, resulting in more and more free shale gas gradually escaping to the earth surface, the atmospheric fresh water on the surface is injected backward into the gas accumulation reservoir at the same time, and when the gas escapes and atmospheric freshwater injection reaches a balance, the gas is re-accumulated in the gas accumulation reservoir of the shale gas reservoir, is classified as the denudation residual type shale gas accumulation mode.

The present invention has the following advantages: by fully absorbing the characteristics of current international and domestic shale gas reservoirs with different enrichment characteristics, detailed analysis is made for the factor construction and the interaction relationship of various factors of a shale gas enrichment system; key principle factors affecting shale gas enrichment and accumulation are extracted; the existing shale gas continuous enrichment and accumulation theory and its characteristic modes are broken through; three major types, six sub-categories of new shale gas enrichment and accumulation modes are established; these modes make the classification of shale gas enrichment and accumulation more elaborate, which is conducive to the development of shale gas exploration and development practices of different scales and technical systems for different shale gas enrichment and accumulation modes. Therefore, the favorable exploration targets of shale gas are refined, the development benefits of the targets can be increased, and the specific implementation targets and directions can be provided while a technical support is provided for the further exploration of shale gas.

### BRIEF DESCRIPTION OF THE DRAWINGS

FIG. 1 is a shale gas enrichment and accumulation system and a classification method thereof;

FIG. 2 is an autochthonous continuous biogenic shale gas enrichment mode pattern;

FIG. 3 is an autochthonous continuous thermogenic shale gas enrichment mode pattern;

FIG. 4 is a positive tectonic accumulation type shale gas enrichment mode pattern;

FIG. 5 is a fractural zone accumulation type shale gas enrichment mode pattern;

FIG. 6 is a fracture-damaged type shale gas accumulation mode pattern;

FIG. 7 is a denudation residual type shale gas accumulation mode pattern;

FIG. 8-1, FIG. 8-2 and FIG. 8-3 are a tectonic shale gas enrichment mode classification and characteristic relationship graph.

### DETAILED DESCRIPTION OF THE EMBODIMENTS

The present invention will be further described below in conjunction with the accompanying drawings, and the protection scope of the present invention is not limited to the followings:

a shale gas enrichment and accumulation classification method comprises the following steps:

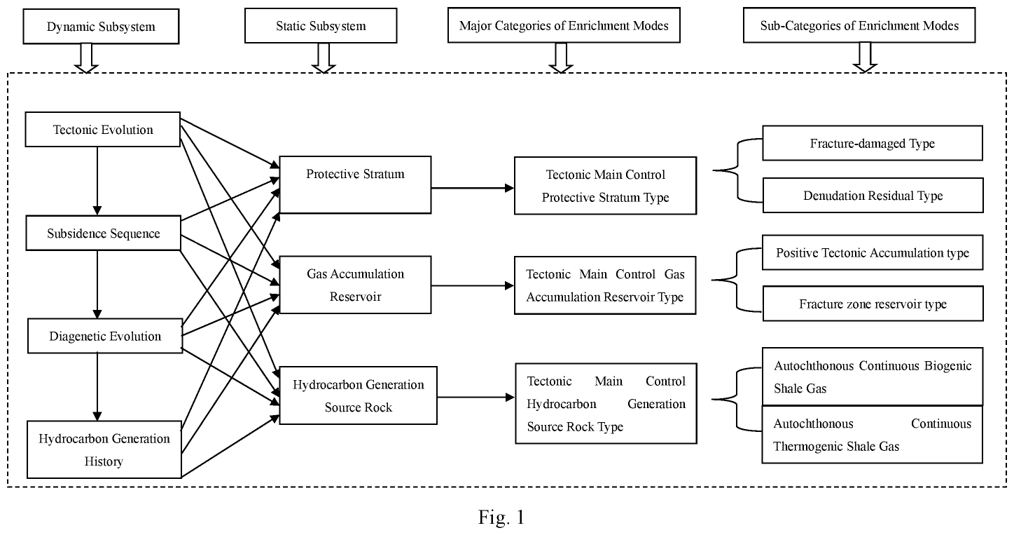

S1. analyzing a shale gas enrichment and accumulation system and dividing the same into three static subsystems of hydrocarbon generation source rock, a gas accumulation reservoir and a protective stratum, as well as four dynamic subsystems of tectonic evolution, sedimentary sequence, diagenetic evolution and hydrocarbon generation history, and establishing three major types, six sub-categories of shale gas enrichment and accumulation modes based on the analysis of the interaction relationship between the dynamic subsystems and the static subsystems and the extraction of principal factors, as shown in FIG. 1;

S2, dividing the shale gas enrichment mode in the step S1 into a tectonic main control hydrocarbon generation source rock type shale gas enrichment mode, a tectonic main control gas accumulation reservoir-type shale gas enrichment mode and a tectonic main control protective stratum type shale gas enrichment mode according to the differences of the degrees of tectonic evolution over shale gas hydrocarbon generation source rock, a gas accumulation reservoir and a protective stratum;

S3, when the tectonic main control hydrocarbon generation rock type shale gas enrichment mode is developed in a stable Cratonic basin or foreland basin; through long-term stable tectonic subsidence, the basin maintains a continuous and stable accommodating space; the resulting deep-water lithofacies palaeogeographic environment of the sea basin is easy to produce a combination of sedimentary sequences of multiple sets of mud shale and compact limestone; these organic matter-rich mud shale and compact limestone or mud shale sedimentary sequences developed at the top of the mud shale become favorable shale gas hydrocarbon generation source rock, a gas accumulation reservoir and a protective stratum, wherein the hydrocarbon generation stage and hydrocarbon-forming products of the hydrocarbon generation source rock are greatly affected by tectonic evolution, dividing the tectonic main control hydrocarbon generation source rock type shale gas enrichment mode into two sub-categories according to the influence of the tectonic evolution of the basin over the hydrocarbon generation stage and hydrocarbon-forming products of the hydrocarbon generation source rock:

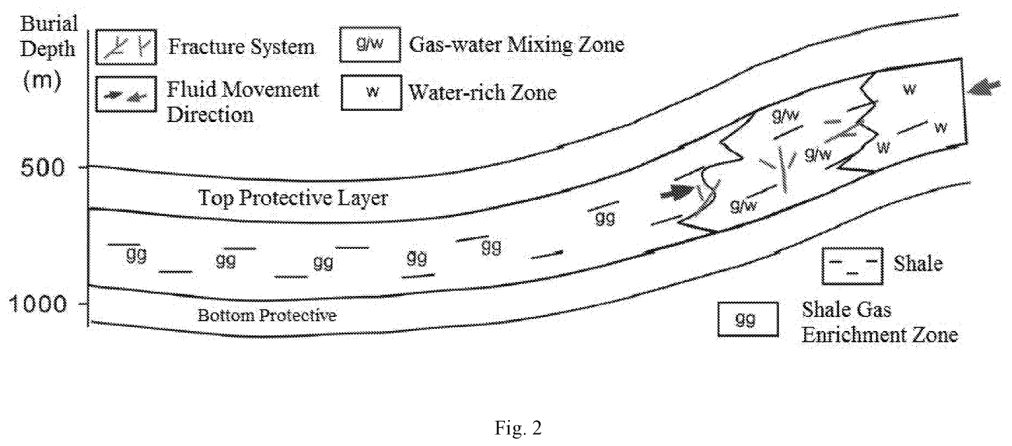

S3(I), an autochthonous continuous biogenic shale gas enrichment mode: a basin in which the tectonic subsidence extent is not large, the buried depth is low, pore water in mud shale is not completely discharged, and meanwhile organic matter-rich bud shale begins to generate biogenetic shale gas in anoxic, low-temperature and watery environments, and is accumulated autochthonously by means of continuous filling of atmospheric fresh water at the edge of the basin, is classified as the autochthonous continuous biogenic shale gas enrichment mode; the specific characteristics are shown in FIG. 2; the characteristics include the typical characteristics, such as the maturity of hydrocarbon source rock and the reservoir pressure are low, but various fractures in the reservoir are developed relatively, the original water saturation is high, the content of adsorbed gas is high, the source-storage-cap system of the gas reservoir is complete, and the enrichment zone is continuously distributed in a large scale;

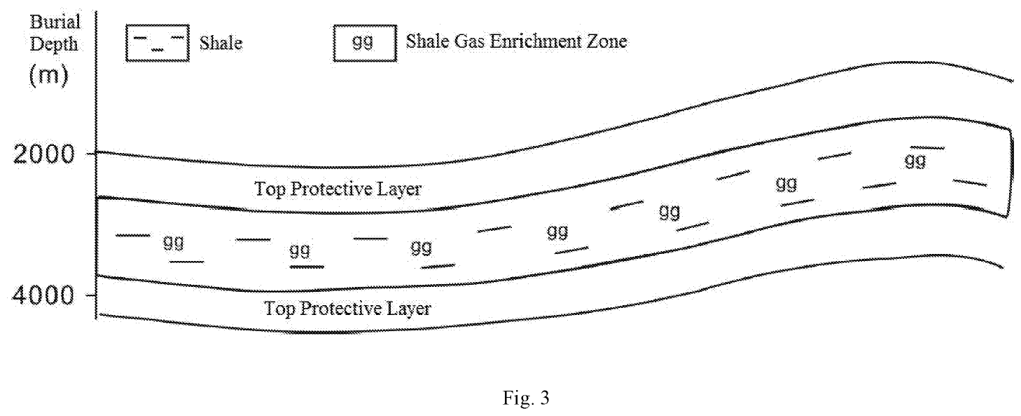

S3(II), an autochthonous thermogenic shale gas enrichment mode: a basin in which the tectonic subsidence extent increases, the buried depth increases, the formation temperature and pressure gradually increase, primary water in pores is gradually discharged by a compaction effect, gradually evaporates under the influence of high temperature and high pressure environments, and is finally exhausted, and kerogen and asphalt organic matters in mud shale begin to form a large amount of hydrocarbons via thermal degradation or thermal cracking, is classified as the autochthonous thermogenic shale gas enrichment mode; the specific characteristics are shown in FIG. 3; the characteristics include the typical characteristics, such as the maturity of hydrocarbon source rock is mid-high, the reservoir pressure is high, but the tectonic fractures in the reservoir are not developed and free of water, the content of adsorbed gas ranges from moderate to low, the source-storage-cap system of the gas reservoir is complete, and the enrichment zone is continuously distributed in a large scale;

in the shale gas enrichment mode of the above two sub-categories, the core of tectonic evolution is embodied in the burial depth formed by tectonic subsidence, which defines the hydrocarbon formation mode of shale organic matters. The formation of a shale reservoir collective pore system and the densification of the top and bottom protective layers thereof are evolved through diagenesis; the spatial distribution of the shale gas reservoir is mainly controlled by the development of the gas accumulation reservoir, thus forming a large-area continuous distribution of shale gas enrichment areas;

S4, when the tectonic main control gas accumulation reservoir type shale gas enrichment mode is developed in a tectonic adjustment zone of the foreland basin; through the rapid subsidence of the basin-forming process in the foreland basin, favorable shale gas hydrocarbon generation source rock and protective stratum sedimentary sequences are formed, which generate a large amount of hydrocarbon and autochthonously drains and enriches hydrocarbon, after entering apyrolytic hydrocarbon generation depth threshold; with the coupled superposition of tectonic movements that occur frequently in the formation of the foreland basin, the spatial from or internal structure of the gas accumulation reservoir undergoes major changes, forcing the previously enriched shale gas to re-adjust and accumulate into a reservoir, dividing the tectonic main control gas accumulation reservoir type shale gas enrichment mode into the following two sub-categories according to the actual characteristics of the tectonic evolution that causes the gas accumulation reservoir to change:

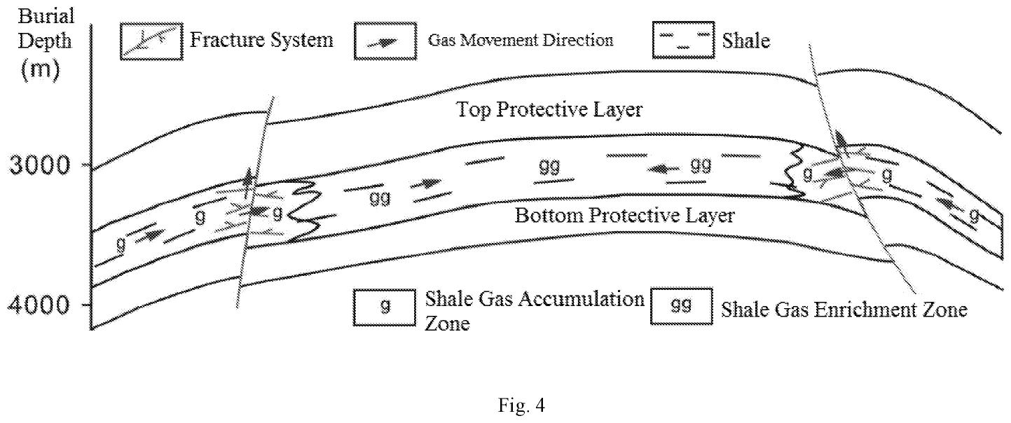

S4(1), a positive tectonic accumulation reservoir-type shale gas enrichment mode: a forelandbasin in which, during the formation process, or during the generation and drainage of a large amount of hydrocarbons of hydrocarbon source rock after formation, an area where the gas accumulation reservoir is located has undergone strong tectonic compression, resulting in large fold deformation of the gas accumulation reservoir that is originally located in a monoclinic structure of the basin slope or in a negative structure in the basin center, is classified as the positive tectonic accumulation reservoir-type shale gas enrichment mode; the specific characteristics are shown in FIG. 4; the characteristics include the typical characteristics, such as the maturity of hydrocarbon source rock is high, the reservoir pressure is ultrahigh, the tectonic fractures at the pleated wing are developed, and the lamellation fractures at the main part of the pleat are opened and free of water, the content of adsorbed gas is moderate, the source-storage-cap system of the gas reservoir is complete, and the shale gas enrichment area is controlled by forward tectonic distribution scale and range;

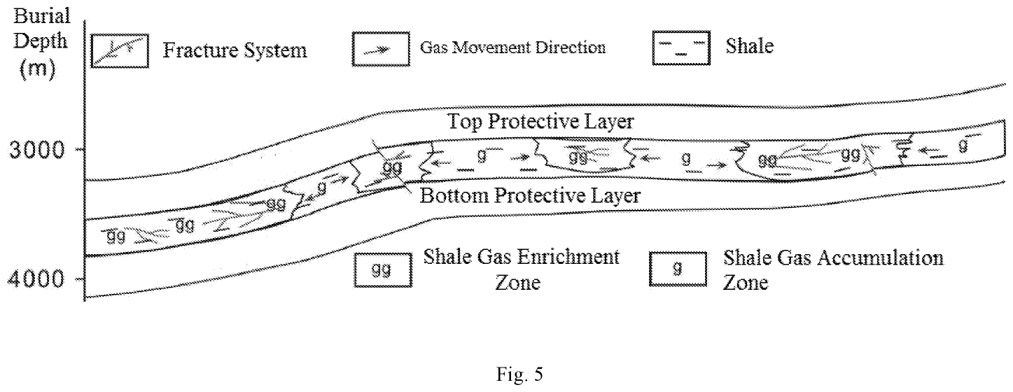

S4(II) a fractural zone accumulation type shale gas enrichment mode: a forelandbasin in which, during the formation process, or during the generation and drainage of a large amount of hydrocarbons of hydrocarbon generation source rock after formation, or at the end of the hydrocarbon generation or drainage process, multiple stages of tectonic lifting movements occur in an area where the gas accumulation reservoir is located, the burial depth of the gas accumulation reservoir results in a sharp fluctuation of the formation temperature and pressure caused by turbulent changes, gas adsorption and desorption processes and free gas shrinkage and expansion processes are repeated continuously to promote the activation of various diagenetic fractures in the gas accumulation reservoir, and the interactive changes of the stress concentration formed by the tectonic lifting movements also induce more tectonic fractures, is classified as the fractural zone accumulation type shale gas enrichment mode; as shown in FIG. 5, the characteristics include the typical characteristics, such as the maturity of hydrocarbon source rock is high, the reservoir pressure is moderate, various rock-forming fractures and tectonic fractures are developed, the content of adsorbed gas is high slightly, the source-storage-cap system of the gas reservoir is complete, and the shale gas enrichment area is controlled by fracture development zone;

in the shale gas enrichment mode of the above two sub-categories, the core of tectonic evolution is embodied in tectonic compression movements or multiple stages of tectonic lifting movements, forcing positive pleat deformation of the shale gas accumulation reservoir, or inducing a fracture development zone therefrom, thereby causing previously accumulated shale gas to be adjusted and enriched, or accumulated to a high pleat position or accumulated to a fracture development zone. Because the strength of the tectonic movements is not large, the top and bottom protective layers of the gas accumulation reservoir are well preserved, and the re-accumulation of shale gas mainly occurs inside the gas accumulation reservoir; the spatial distribution of the shale gas reservoir is mainly controlled by the distribution scale of the positive structure or the fracture development zone in the gas accumulation reservoir, resulting in a continuous distribution of the shale gas enrichment area only within a certain area;

S5, dividing a tectonic main control protective stratum type shale gas accumulation mode into the following two sub-categories:

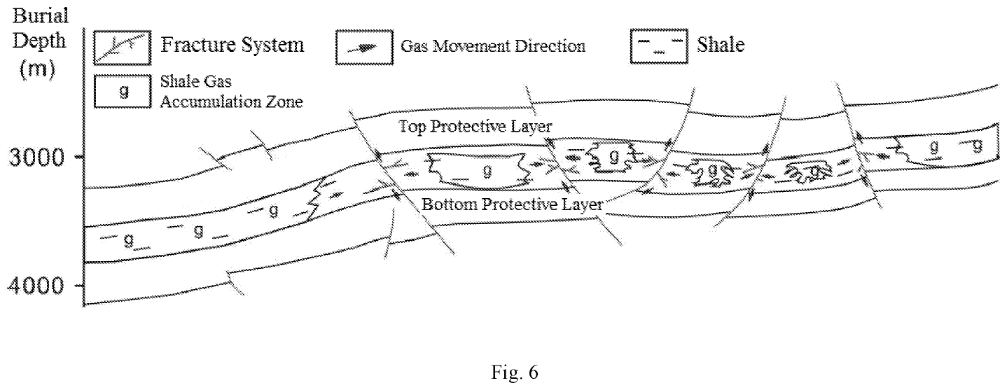

S5(I), a fracture-damaged type shale gas accumulation mode: a shale gas reservoir in which, after undergoing multi-stage tectonic compression or tensile action, various types of extruded or tensile faults and induced fractures thereof begin to occur in the core area of the shale gas reservoir, the original gas accumulation reservoir and top and bottom protective strata thereof begin to be cut by many fault blocks, the shale gas enriched near the faults gradually dissipates to relief pressure along the faults and the induced fractures thereof, causing the cutting damage of the original shale gas reservoir, the shale itself is dense and has a certain covering capability, and in addition to the existence of the top and bottom protective layers thereof, the locally accumulated shale gas remains in the fault blocks away from the faults and the induced fractures, is classified as the fracture-damaged type shale gas accumulation mode; as shown in FIG. 6, the characteristics include the typical characteristics, such as the faults and fractures in the core zone of the shale gas reservoir are developed, the maturity of hydrocarbon source rock is high, and the reservoir pressure is low, the content of adsorbed gas is low, the source-storage-cap system of the gas reservoir is broken and destroyed, and the shale gas enrichment area is controlled by the scale of single fault block;

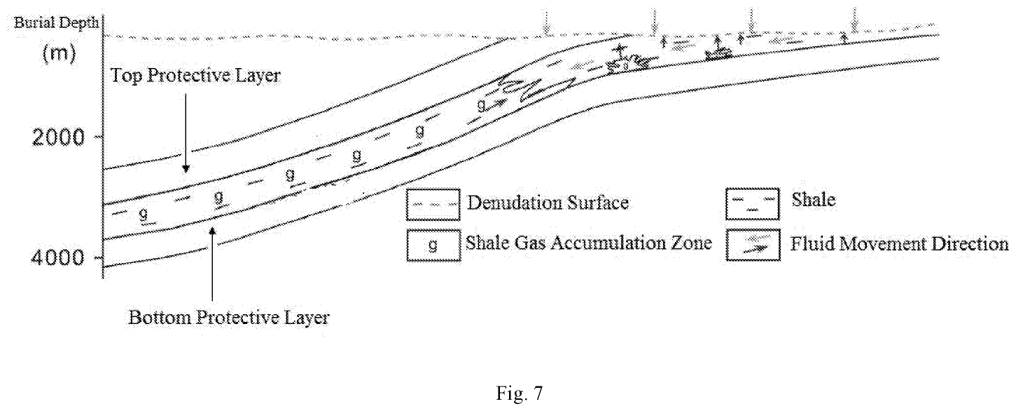

S(II), a denudation residual type shale gas accumulation mode: a shale gas reservoir in which, after undergoing multiple stages of tectonic uplifting movements, the core area of the shale gas reservoir will continue to rise, the dip angle of the formation becomes larger, causing the updraft ends of the gas accumulation reservoir and the top and bottom protective strata thereof to be exposed out of the earth surface and suffer from the leaching effect of surface atmospheric water, shale gas enriched therein is adsorbed to form a large amount of free gas because of depressurization and desorption, N2 and CO2 from air displaces a large amount of shale gas due to stronger adsorption on the other hand after entering into the shale reservoir, resulting in more and more free shale gas gradually escaping to the earth surface, the atmospheric fresh water on the surface is injected backward into the gas accumulation reservoir at the same time, and when the gas escapes and atmospheric freshwater injection reaches a balance, the gas is re-accumulated in the gas accumulation stratum of the shale gas reservoir, is classified as the denudation residual type shale gas accumulation mode; as shown in FIG. 7, this mode has the typical characteristics: the gas accumulation reservoir and the top and bottom protective strata thereof are not complete, the maturity of hydrocarbon source rock is high, and the reservoir pressure is low, the content of adsorbed gas is low, the reservoir contains a large amount of water, and the shale gas enrichment area is controlled by the scales of the residual gas accumulation reservoir and the top and bottom protective strata thereof;

in the shale gas accumulation mode of the above two sub-categories, the core of tectonic evolution is embodied in the strong transformation of multiple stages of tectonic movements, causing the breakage or erosion of the original shale gas reservoir; the gas accumulation reservoir and the top and bottom protective strata thereof are fractured or cut, or subjected to denudation, resulting in re-accumulation of shale gas, formation of a secondary fault block-type shale gas reservoir with a significantly reduced enrichment level, or denudation of the residual shale gas reservoir; the spatial distribution of the shale gas reservoir is mainly controlled by the development level of the faults or the distribution characteristics of the denuded areas, resulting in a sporadic distribution of shale gas accumulation areas.

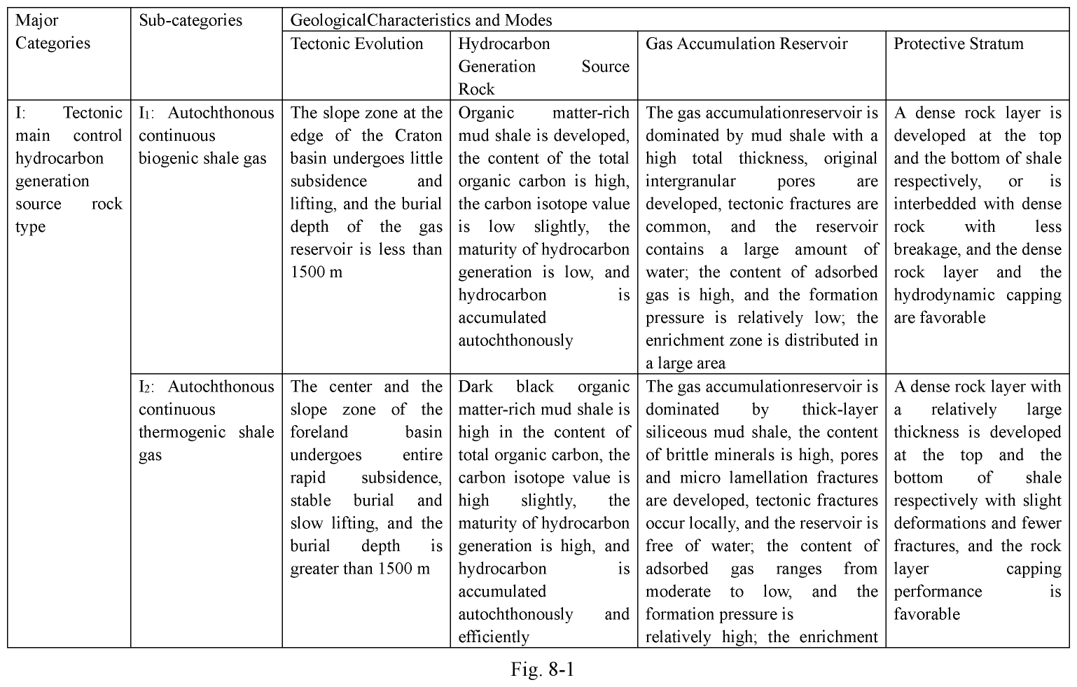

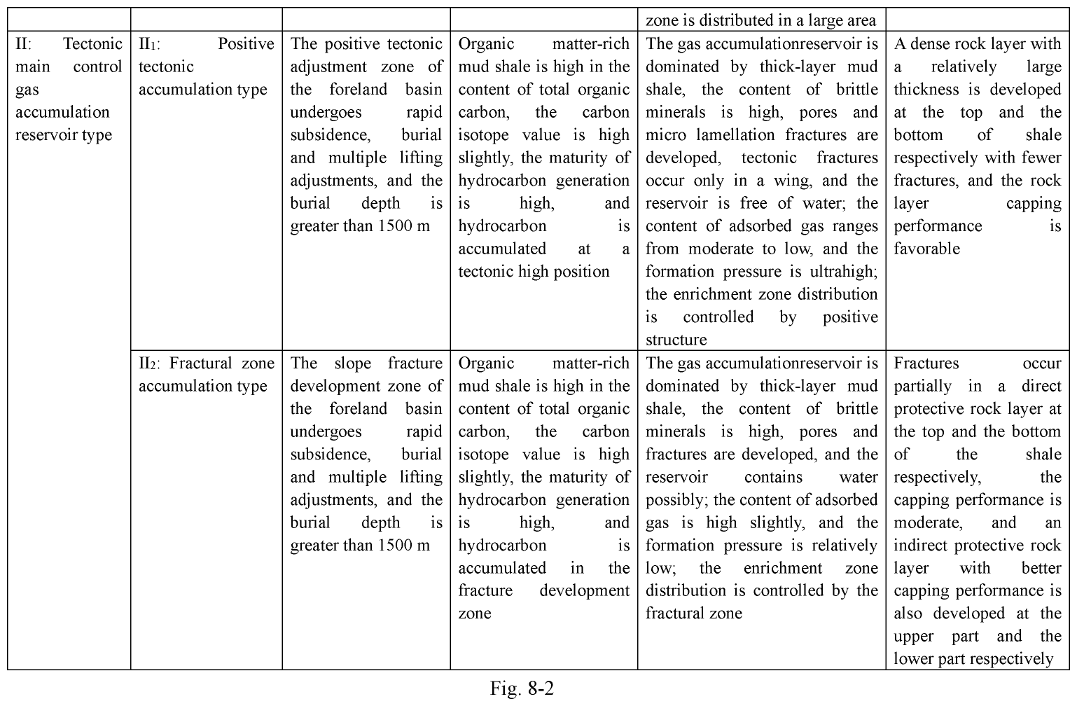

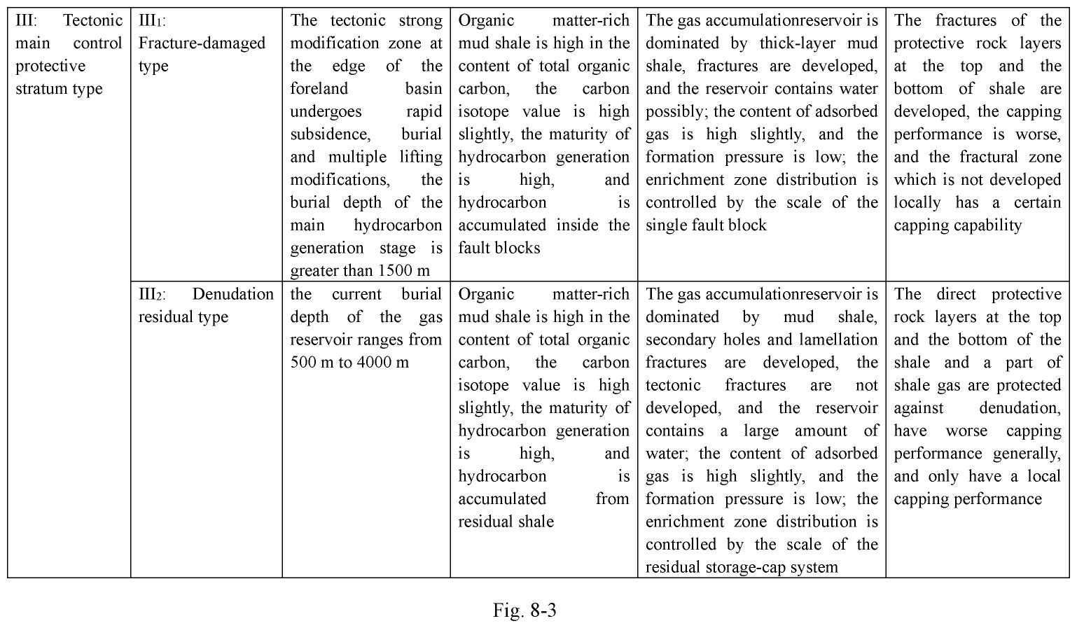

Therefore, through the classification steps of steps S1 to S5, as shown in FIG. 8-1, FIG. 8-2 and FIG. 8-3, a classification and characteristic table of the tectonic shale gas enrichment mode is created.

Therefore, by fully absorbing the characteristics of current international and domestic shale gas reservoirs with different enrichment characteristics, detailed analysis is made for the factor construction and the interaction relationship of various factors of a shale gas enrichment system; key principle factors affecting shale gas enrichment and accumulation are extracted; the existing shale gas continuous enrichment and accumulation theory and its characteristic modes are broken through; three major types, six sub-categories new shale gas enrichment and accumulation modes are established; these modes make the classification of shale gas enrichment and accumulation more elaborate, which is conducive to the development of shale gas exploration and development practices of different scales and technical systems for different shale gas enrichment and accumulation modes. Therefore, the favorable exploration targets of shale gas are refined, the development benefits of the targets can be increased, and the specific implementation targets and directions can be provided while a technical support is provided for the further exploration of shale gas.

The above contents are only preferred embodiments of the present invention, however, it should be understood that the present invention is not limited to the forms disclosed herein, and is not to be construed as the exclusion of the other embodiments, but may be used in various other combinations, modifications and environments and may be modified by the above teachings or related art or knowledge within the conception scope herein. All changes and modifications made by those skilled in the art are intended to be within the protection scope of the appended claims.

1. A shale gas enrichment and accumulation classification method, comprising the following steps:
S1: analyzing a shale gas enrichment and accumulation system and dividing the shale gas enrichment and accumulation system into three static subsystems and four dynamic subsystems, wherein the three static subsystems comprise a hydrocarbon generation source rock, a gas accumulation reservoir and a protective stratum, and the four dynamic subsystems comprise a tectonic evolution, a sedimentary sequence, a diagenetic evolution and a hydrocarbon generation history, and establishing three major types and six sub-categories of shale gas enrichment and accumulation modes based on an analysis of an interaction relationship between the four dynamic subsystems and the three static subsystems and an extraction of principal factors;
S2: dividing the shale gas enrichment and accumulation modes into a tectonic main control hydrocarbon generation source rock type shale gas enrichment mode, a tectonic main control gas accumulation reservoir-type shale gas enrichment mode and a tectonic main control protective stratum type shale gas enrichment mode according to differences of tectonic evolution degrees on the shale gas hydrocarbon generation source rock, the gas accumulation reservoir and the protective stratum;
S3: dividing the tectonic main control hydrocarbon generation source rock type shale gas enrichment mode into two first sub-categories, wherein the two first sub-categories comprise:
an autochthonous continuous biogenic shale gas enrichment mode: a basin in which a tectonic subsidence extent is not large, a buried depth is low, pore water in mud shale is not completely discharged, and an organic matter-rich bud shale begins to generate biogenetic shale gas in anoxic, low-temperature and watery environments, and is accumulated autochthonously by means of continuous filling of atmospheric fresh water at an edge of the basin, is classified as the autochthonous continuous biogenic shale gas enrichment mode; and
an autochthonous thermogenic shale gas enrichment mode: the basin in which the tectonic subsidence extent increases, the buried depth increases, a formation temperature and pressure gradually increase, primary water in pores is gradually discharged by a compaction effect, gradually evaporates under an influence of high temperature and high pressure environments, and is finally exhausted, and kerogen and asphalt organic matters in the mud shale begin to generate a large amount of hydrocarbons via thermal degradation or thermal cracking, is classified as the autochthonous thermogenic shale gas enrichment mode;
S4: dividing the tectonic main control gas accumulation reservoir-type shale gas enrichment mode into two second sub-categories, wherein the two second sub-categories comprise:
a positive tectonic accumulation reservoir-type shale gas enrichment mode: a forelandbasin in which, during a formation process, or during a generation and drainage process of a large amount of hydrocarbons of hydrocarbon generation source rock after formation, an area where the gas accumulation reservoir is located has undergone strong tectonic compression, resulting in large fold deformation of the gas accumulation reservoir that is originally located in a monoclinic structure of a basin slope or in a negative structure in a basin center, is classified as the positive tectonic accumulation reservoir-type shale gas enrichment mode; and
a fractural zone accumulation type shale gas enrichment mode: the forelandbasin in which, during the formation process, or during the generation and drainage process of the large amount of hydrocarbons of the hydrocarbon generation source rock after the formation, or at the end of the hydrocarbon generation or drainage process, multiple stages of tectonic lifting movements occur in the area where the gas accumulation reservoir is located, the buried depth of the gas accumulation reservoir results in a sharp fluctuation of the formation temperature and pressure caused by turbulent changes, gas adsorption and desorption processes and free gas shrinkage and expansion processes are repeated continuously to promote an activation of various diagenetic fractures in the gas accumulation reservoir, and an interactive changes of the stress concentration formed by the tectonic lifting movements also induce more tectonic fractures, is classified as the fractural zone accumulation type shale gas enrichment mode;
S5: dividing a tectonic main control protective stratum type shale gas enrichment mode into the following two third sub-categories, wherein the two third sub-categories comprise:
a fracture-damaged type shale gas accumulation mode: a shale gas reservoir in which, if undergoing multi-stage tectonic compression or tensile action, various types of extruded or tensile faults and induced fractures of the extruded or tensile faults begin to occur in a core area of the shale gas reservoir, an original gas accumulation reservoir and a top and bottom protective strata of the original gas accumulation reservoir begin to be cut by many fault blocks, shale gas enriched near faults gradually dissipates to relief pressure along the faults and the induced fractures of the extruded or tensile faults, causing a cutting damage of the original shale gas reservoir, shale is dense and has a certain covering capability, and in addition to an existence of the top and bottom protective strata, the shale gas that is locally accumulated remains in the fault blocks away from the faults and the induced fractures, is classified as the fracture-damaged type shale gas accumulation mode; and
a denudation residual type shale gas accumulation mode: if the shale gas reservoir in which, after undergoing multiple stages of tectonic uplifting movements, the core area of the shale gas reservoir will continue to rise, a dip angle of the formation becomes larger, causing an updraft ends of the gas accumulation reservoir and the top and bottom protective strata of the gas accumulation reservoir to be exposed out of an earth surface and suffer from a leaching effect of atmospheric fresh water on the surface, the shale gas enriched from the shale gas reservoir is adsorbed and becomes a large amount of free gas because of depressurization and desorption, N2 and CO2 from air displaces a large amount of the shale gas due to stronger adsorption after entering into the shale gas reservoir, resulting in more and more free shale gas gradually escaping to the earth surface, the atmospheric fresh water on the earth surface is injected backward into the gas accumulation reservoir at the same time, and when the shale gas escapes and an injection of the atmospheric fresh water reaches a balance, the shale gas is re-accumulated in the gas accumulation reservoir of the shale gas reservoir, is classified as the denudation residual type shale gas accumulation mode.
